(19) United States
(12) Patent Application Publication
(10) Pub. No.: US 2026/0026777 A1
(43) Pub. Date: Jan. 29, 2026

(54) METHOD AND DEVICE FOR LOCALLY ESTIMATING THE SPEED OF SOUND IN A REGION OF INTEREST OF A MEDIUM

(71) Applicant: E-SCOPICS, Aix-en-Provence (FR)

(72) Inventors: Adrien BESSON, Marseille (FR); Baptiste HERIARD-DUBREUIL, Aix-en-Provence (FR); Frederic WINTZENRIETH, Aix-en-Provence (FR)

(21) Appl. No.: 18/845,724

(22) PCT Filed: Mar. 10, 2023
(86) PCT No.: PCT/EP2023/056234

§ 371 Date: Mar. 11, 2025

(30) Foreign Application Priority Data
Mar. 10, 2022 (FR) 2202104

(51) Int. Cl. A61B 8/08; A61B 8/00

(52) U.S. Cl. CPC A61B 8/08; A61B 8/4488

(57) ABSTRACT

The present invention relates to a method for analysing a medium using an array of transducers (T1-Tn), the method comprising: —acquiring (100) two reception signals each associated with a respective pair of waves transmitted and received according to transmission and reception directions; —deforming (200) the two reception signals to obtain two deformed signals, the deformation step comprising, for each reception signal, composition of the reception signal with an affine function dependent on half the angular difference between the transmission and reception directions of the pair associated with the reception signal; —locally estimating (300) the speed of sound in a region of interest of the medium based on the comparison of the two deformed signals.

### FIELD OF THE INVENTION

The present invention relates to the general technical field of the analysis of a medium by propagation of waves, and in particular sonic or ultrasonic waves, or electromagnetic waves.

More specifically, the present invention relates to a method and a device for the local estimation of the speed of sound in a region of interest of a target object, or of a diffuse medium such as a biological, human or animal tissue.

In the following, the present invention will be described with reference to medical imaging by ultrasound, it being understood that the teachings described here can be used in other types of applications (non-medical ultrasound, SONAR, RADAR, etc.) using waves whose amplitude, frequency and phase are controllable (i.e., coherent waves).

### BACKGROUND OF THE INVENTION

Hepatic steatosis is a disease characterized by excessive accumulation of hepatic fat (i.e., fat inside the liver). It can develop into fibrosis and then into hepatic cirrhosis.

A technique based on the magnetic resonance imaging (MRI) can be used to detect steatosis in a patient. Indeed, MRI allows measuring the fat fraction in proton density as a biomarker of the hepatic fat content. However, the MRI is not widely available and is expensive.

Therefore, ultrasound-based techniques have been developed to quantify hepatic fat.

More specifically, an imaging probe adapted to emit ultrasound waves and detect backscattered waves is used to acquire signals corresponding to said backscattered waves. These signals are then transmitted to a processing unit to assess the speed of sound in the liver in order to determine the accumulation of fat inside the liver.

Indeed, the speed of sound inside a tissue varies as a function of the amount of fat it contains. In particular, a steatotic liver has a speed of sound slightly lower (typically 1,460 m/s) than that of a healthy liver (typically 1,580 m/s).

However, since this decrease in the speed of sound is small (5 to 10% variation in the speed of sound between a healthy liver and a steatotic liver), it can be difficult to identify a speed of sound characteristic of hepatic steatosis.

This is why the speed of sound must be estimated as accurately as possible to detect hepatic steatosis.

However, the liver is located inside the body. It is therefore covered with skin, fat and muscles. These different tissues—located between the probe and the liver—can disrupt the estimation of the speed of sound in the liver.

Currently, most of the techniques developed based on ultrasound do not allow a local measurement of the speed of sound in the liver. Thus, the speed of sound estimated with such techniques corresponds to the speed of sound between the upper surface of the skin and the liver. The diagnosis established by the practitioner is then highly dependent on his experience with the device used, since the speed of sound estimated by the device does not correspond to the local speed of sound in the liver.

This is why other ultrasound-based solutions have been developed to estimate the local speed of sound in the liver. Document WO 2020/070104 describes for example a method for estimating the local speed of sound in the liver comprising the following steps:
determining on a morphological image a position of at least one interface in the medium, the interface dividing the medium into an intermediate region and a target region—namely the patient's liver—in the depth direction,
determining a first speed of sound of the intermediate region based on signals detected by the probe, and
determining the target speed of sound within the target region—namely the patient's liver—based on at least some of the detected signals and by taking into account the position of the interface and the first speed of sound determined.

Thus in these solutions, the speed of sound in the tissues located between the probe and the liver (i.e., skin, fat and muscle) is calculated to deduce the local speed of sound in the liver.

Another solution is described in document WO 2015/091519 which concerns a method for determining a local speed of sound in an object. This method comprises the following steps:
transmitting:
   a first ultrasound pulse in a first direction,
   a second ultrasound pulse in a second direction different from the first direction,
detecting:
   first echoes corresponding to the backscattering of the first ultrasound pulse in the object,
   second echoes corresponding to the backscattering of the second ultrasound pulse in the object,
reconstructing:
   from the first echoes detected, a first associated image of first local echoes,
   from the second echoes detected, a second associated image of second local echoes,
said images being located in an image plane extending in the first and second directions,
determining, from the first and second images, a local echo phase shift corresponding to a difference in an echo duration between a first local echo and a corresponding second local echo, relative to the case of an assumed constant speed of sound in the object,
determining the local speed of sound in the object for at least one region of the image plane in the object from the local echo phase shift determined in the previous step.

Such a solution, while allowing an accurate estimation of the local speed of sound in the liver, requires the acquisition of numerous signals as well as the implementation of numerous calculations that are costly in terms of hardware and software resources.

These solutions are not direct, in the sense that they require obtaining information representative of the integrated speed of sound (or average speed of sound through all the tissues crossed by the ultrasonic wave), then apply an algorithm for finding the local speed of sound in the region of interest, such as the patient's liver.

One aim of the present invention is to propose an analysis method and device for directly estimating the local speed of sound of a region of interest such as a patient's liver.

More specifically, one aim of the present invention is to propose an analysis method and device that do not require calculating the speed of sound in the intermediate tissues located between an acquisition probe and a region of interest in order to estimate the speed of sound in this region of interest.

### BRIEF DESCRIPTION OF THE INVENTION

For this purpose, the invention proposes a method for analyzing a medium from an array of transducers, said method comprising:
the acquisition of at least two reception signals each associated with a respective pair of emitted and received waves, said at least two reception signals comprising:
   a first reception signal associated with a first pair of emitted and received waves along first emission and reception directions,
   a second reception signal associated with a second pair of emitted and received waves along second emission and reception directions,
the first emission and reception directions being different from the second emission and reception directions, the acquisition step including, for each reception signal, the following sub-steps:
   generating in a scattering medium, by transducers of the array, an emitted wave having a desired emission direction,
   receiving, by transducers of the array, reverberated signals and their combinations to obtain a time reception signal representative of a received wave reflected by the scattering medium along a desired reception direction,
the distortion of said at least two reception signals by composition with a respective affine function:
   the first reception signal being distorted by composition with a first affine function to obtain a first distorted signal, the first affine function depending on the first emission and reception directions,
   the second reception signal being distorted by composition with a second affine function, different from the first affine function, to obtain a second distorted signal, the second affine function depending on the second emission and reception directions,
the local estimation of the speed of sound in a region of interest of the medium from the comparison of the first and second distorted signals.

Thus, the method according to the invention differs from the prior art in that it makes it possible to obtain a map and/or a local measurement of the speed of sound in a region of interest directly and with the following advantages:
the method provides a local measurement in a region of interest (map or value),
it requires very few ultrasonic emissions (typically between two and sixteen) compared to the other local measurement techniques (typically more than a hundred),
it requires a very limited amount of calculation unlike the other non-direct local measurement techniques that require inverting matrices or storing large pre-inverted matrices and applying them,
it requires a very limited amount of data derived from the ultrasonic probe (works from two signals where the other methods generally use 100*128 signals).

Preferred but non-limiting aspects of the invention are the following:
the estimation step can comprise the following sub-steps:
   determining a transformation coefficient between the first and second distorted signals, said transformation coefficient being representative of a difference in time scale between the first and second distorted signals,
   obtaining the speed of sound in the region of interest of the medium from the transformation coefficient;
the estimation step can comprise a sub-step of calculating a resulting signal representative of an average correlation between the first and second distorted signals;
the average correlation can be obtained by at least one of the following methods:
   averaging over different depths in the region of interest,
   averaging over different media with the same speed of sound,
   averaging over signals obtained with different emission or reception strategies,
   averaging over different signals obtained by disturbing delay laws applied to the array of transducers in emission or in reception;
the transformation coefficient can be determined by derivation of a phase of the resulting signal representative of the average correlation between the first and second distorted signals;
the transformation coefficient D can be determined from the following formula:

$$D := \frac{\partial ∠ 〈 \hat{s} ( t ) {\hat{{s}^{′}} ( t )}^{*} 〉}{\partial t}$$

where:
   [symbol] ŝ(t) [symbol] (t)* [symbol] corresponds to the average correlation of the first and second distorted signals ŝ and ŝ′,

$$\frac{\partial ∠}{\partial t}$$

is the operator of the time derivation of the phase;
the sub-step of obtaining the speed of sound in the region of interest can include the resolution of the following formula:

$${c}_{r} = {c}_{th} \sqrt{1 - \frac{\frac{2}{{ω}_{c}} D}{{{δ}_{th}^{′}}^{2} - {δ}_{th}^{2}}} ,$$

where:
   “cᵣ” is the speed of sound in the region of interest,
   “cₜₕ” is a theoretical speed of sound used to calculate the first and second affine functions during the distortion step,
   “D” is the transformation coefficient,
   “ω_{c}” is a central pulse of the signals s and s′ at the considered depth,
   “δ” is a half-angular difference between the first emission and reception directions of the first pair of emitted and received waves,
   “δ′” is a half-angular difference between the second emission and reception directions of the second pair of emitted and received waves;
each affine function can be of the type:

$$f ( t ) = A t + B ,$$

where:
   “A” is a leading coefficient depending on the half-angular difference between the emission direction and the reception direction of the pair of emitted and received waves associated with the reception signal, and
   “B” is a factor of alignment of the reception signals at a desired depth corresponding to the depth of the region of interest;
the method can comprise a step of determining the alignment factor “B”, said determination step comprising the sub-steps consisting in:
   acquiring two primary signals each associated with a respective pair of emitted and received waves along emission and reception directions, each primary signal representing an amplitude as a function of a time variable,
   temporally widening each primary signal by application of a factor to the time variable to obtain an enlarged primary signal, the factor depending on the half-angular difference between the emission direction and the reception direction of the pair associated with the primary signal,
   correlating the enlarged primary signals,
   deducing from the phase of the correlation an optimal offset index B;
for each reception signal, the sub-step of combining the reverberated signals can comprise the summation of the reverberated signals according to a respective delay law, said respective delay laws being defined so that the bisectors of the directions of the emission and reception waves of each pair of emitted and received waves coincide;
the method can further comprise a step of estimating the central pulse of the signals s and s′ at the considered depth, said estimation step comprising the following sub-steps:
   estimating the central pulse of the first reception signal s from its autocorrelation,
   estimating the central pulse of the second reception signal from its autocorrelation,
   estimating a final central pulse from the average of the central pulses of each of the reception signals.

### BRIEF DESCRIPTION OF THE DRAWINGS

Other advantages and characteristics of the analysis method and the device according to the invention will emerge more clearly from the following description of several variants of embodiments, given as non-limiting examples, from the appended drawings in which:

FIG. 1 is a schematic representation of an analysis method for estimating a speed of sound,

FIG. 2 is a schematic representation of an ultrasound imaging device including an acquisition probe and one (or more) calculation unit(s),

FIG. 3 is a schematic representation of plane, spiral and divergent waves emitted by planar and curved arrays of transducers,

FIG. 4 is a schematic representation illustrating the principle of emission of a plane wave from an array of transducers,

FIG. 5 is a schematic representation of waves emitted and reverberated by a medium for three different instants,

FIG. 6 is a schematic representation illustrating two pairs of emission and reception angles,

FIGS. 7a to 7f illustrate examples of emission and reception of pairs of emission and reception waves,

FIG. 8 is a schematic sectional representation of a medium to be imaged,

FIG. 9 is a graph illustrating a phase of a correlation between two reception signals associated with different pairs of emission and reception ultrasonic waves (pair (10°, −10°) and pair (0°, 0°)) as a function of time,

FIG. 10 is a graph illustrating an expansion factor as a function of time,

FIG. 11 is a graph illustrating a speed of sound as a function of time,

FIG. 12 schematically illustrates the propagation of a non-zero angle emission wave at an interface between two layers of a medium to be imaged.

### DETAILED DESCRIPTION OF THE INVENTION

Different embodiments of the method and device for analyzing a medium according to the invention with reference to the figures will now be described in more detail. In these different figures, the equivalent elements are designated by the same numerical reference.

In the following, the invention will be described with reference to the field of imaging of the human body by ultrasound scan. It is obvious to those skilled in the art that the method and device for analyzing a medium according to the invention can be used for other applications, such as SONAR, RADAR applications, or other non-medical applications (seismography, study of materials such as concrete or polycrystalline materials, etc.).

### 1. Presentation

The analysis method and device described below allow a direct estimation of the local speed of sound of a region of interest of a scattering medium, such as the liver of a patient, by ultrasound imaging.

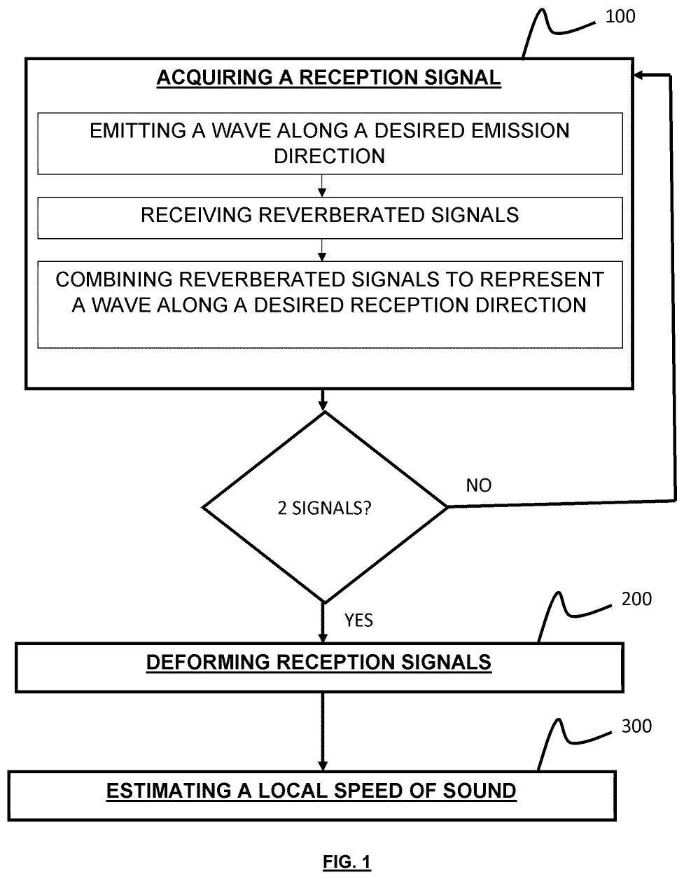

With reference to FIG. 1, the analysis method comprises the following steps:
acquiring 100 at least the first and second reception signals, said step of acquiring the first (respectively second) reception signal comprising the following sub-steps:
   emitting in the medium a first (respectively second) emitted ultrasonic wave in a first (respectively second) emission direction of angle α (respectively α′ different from the angle α), and
   receiving signals reverberated by the medium, and their combination to obtain the first (respectively second) reception signal along a first (respectively second) reception direction of angle β (respectively β′ different from β),
distorting 200 each of said first and second reception signals, by composition with a respective affine function (first affine function for the first reception signal, and second affine function for the second reception signal), to obtain first and second distorted signals,
directly estimating 300 a local speed of sound by comparison of the first and second distorted signals.

Advantageously, in addition to the combination of the signals reverberated by the medium, the reception sub-step can comprise a windowing of the reverberated signals. More specifically, each reverberated signal is multiplied by a function (i.e., observation window function), such as a rectangular function h(t) defined such that:

$$h ( t ) = \{ 1 if t ∈ [ {T}_{1} ; {T}_{2} ] 0 otherwise .$$

Thus, the reverberated signals can be truncated (i.e., windowed) over a time window of duration Δt centered around a time t (which can for example correspond to a flight time of the ultrasonic wave to reach a depth of interest in the medium). The first and second reception signals derived from the combination of the truncated reverberated signals are representative of the medium over a period of time (of a duration comprised between T₁-T₂) desired for the observation of said medium.

Moreover, the first and second reception directions of angles β and β′ can advantageously be chosen so that the bisector of the first emission and reception directions coincides with the bisector of the second emission and reception directions (i.e., the angles (α+β)/2 and (α′+β′)/2 are equal), as will become clear below.

### 2. Device for Analyzing a Medium

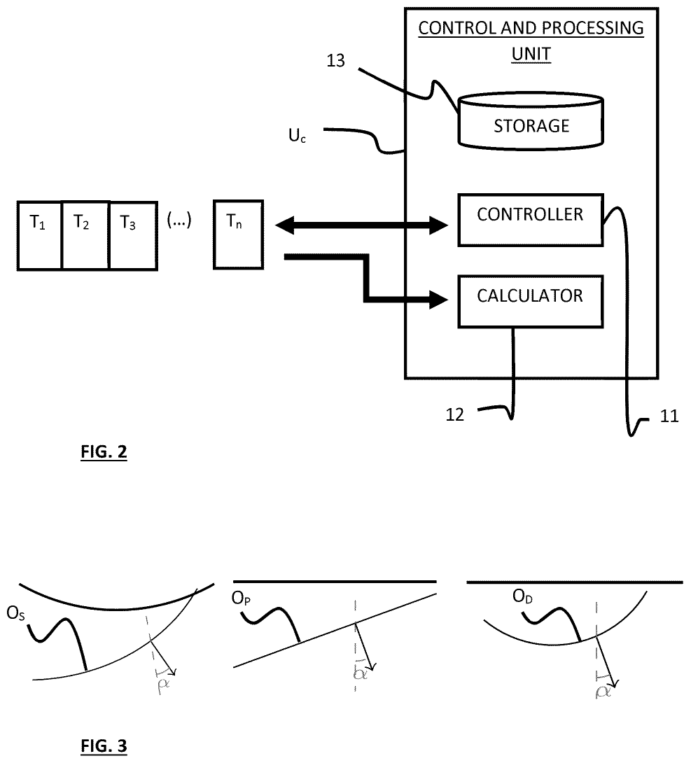

Referring to FIG. 2, one example of a device is illustrated in which the method for analyzing a medium described below can be implemented.

This device comprises:
an array of transducers T₁-Tₙ for the signal acquisition, and
a control and processing unit U_{c} for:
   driving the array of transducers T₁-Tₙ and
   processing the signals acquired by the array of transducers T₁-Tₙ.

### 2.1. Array of Transducers

The array of transducers T₁-Tₙ comprises a set of “n” ultrasonic transducers (“n” being an integer greater than or equal to one) disposed linearly. As a variant, the transducers T₁-Tₙ of the array can be disposed in a curve, or in concentric circles, or in a matrix.

The array of transducers T₁-Tₙ makes it possible to emit excitation ultrasonic waves towards a medium to be analyzed (organ, biological tissue, etc.), and to receive acoustic echoes (i.e., ultrasonic waves reflected by the medium to be analyzed).

Each transducer T₁-Tₙ consists, for example, in a rectangular-shaped piezoelectric material wafer coated on its front and rear faces with electrodes and covered on the front face with lenses and acoustic impedance adaptation layers. Such transducers are known to those skilled in the art and will not be described in more detail below.

In the variant of embodiment illustrated in FIG. 2, all the transducers T₁-Tₙ of the array are used in both emission and reception. In other embodiments, distinct transducers can be used for the emission and reception.

### 2.2. Control and Processing Unit

The control and processing unit U_{c} is connected to the array of transducers T₁-Tₙ.

It allows driving the transducers T₁-Tₙ of the array, and processing the data acquired by the transducers T₁-Tₙ of the array.

More specifically, the control and processing unit U_{c} allows:
ordering the transducers T₁-Tₙ to emit ultrasonic waves towards the medium to be analyzed,
ordering the transducers T₁-Tₙ to receive echoes reflected by the medium to be analyzed and convert them into reception signals,
processing the reception signals.

The control and processing unit U_{c} can be composed of one or more distinct physical entities, possibly remote from the array of transducers T₁-Tₙ.

The control and processing unit U_{c} comprises for example:
one (or more) controller(s) 11, such as a Smartphone, a personal digital assistant (or PDA), or any type of mobile terminal known to those skilled in the art; and
one (or more) calculator(s) 12, such as one or several computer(s), one or several microcomputer(s), one or several workstation(s), and/or other devices known to those skilled in the art including one or several processor(s), one or several microcontroller(s), one or several programmable logic controller(s), one or several application-specific integrated circuit(s), and/or other programmable circuits,
one (or more) storage unit(s) 13 including one (or more) memory(ies) which can be a ROM/RAM memory, a USB key, a memory of a central server.

In addition to retaining data associated with the analysis of a medium, the storage unit 13 also makes it possible to store programming code instructions intended to execute the steps of the analysis method described below.

### 3. Analysis Method

### 3.1. Generalities

One of the advantageous aspects of the analysis method according to the invention relates to the use of highly angular waves in emission and reception, such as the plane, spiral, divergent, or weakly focused waves. In addition, these waves can be two-dimensional or three-dimensional.

Such waves are illustrated in FIG. 3. More specifically, FIG. 3 illustrates:
a plane wave O_{P} emitted by a planar array of transducers T_{P},
a spiral wave O_{S} emitted by a curved array of transducers T_{C},
a divergent wave O_{D} emitted by a curved array of transducers T_{C}.

It is obvious to those skilled in the art that the type of wave is independent of the shape of the array of transducers. Particularly, a planar array of transducers can be configured to emit a spiral wave or a divergent wave (by using a suitable delay law). Similarly, a curved array of transducers can be configured to emit a plane wave (by using a suitable delay law).

The direction of such a wave is defined as its propagation direction in a region of interest. This direction can be characterized by an angle defined relative to a reference direction. For a pair of emitted and received waves, the bisector of the directions can thus be defined as the average between the direction of the emitted wave and that of the received wave. The half-angular difference between the emission and reception directions can also be defined as half the angle formed by the emission and reception directions.

The different steps of the analysis method will now be described in more detail, and particularly the steps of:
emitting in a scattering medium at least:
   a first emitted ultrasonic wave propagating in a first direction of emission angle α, and
   a second emitted ultrasonic wave propagating in a second direction of emission angle α′,
acquiring at least:
   a first reception signal representative of a first received wave propagating along a first reception direction having a first reception angle,
   a second reception signal representative of a second received wave propagating along a second reception direction having a second reception angle different from the first reception angle,
distorting each of the first and second reception signals, by composition with an affine function, to obtain first and second distorted signals, and
directly estimating a local speed of sound from the first and second distorted signals.

For the sake of simplicity, these steps will be described in the case of plane waves, with a set of “n” ultrasonic transducers disposed linearly. This method can however be generalized to other transducer geometries and other types of waves (in particular spiral or divergent waves).

### 3.2. Emission

In a first step, the analysis method comprises the emission of a plurality (two or more than two) of emitted ultrasonic waves each having a respective emission angle different from the emission angles of the other emitted waves of the plurality of emitted ultrasonic waves.

More specifically, for each emitted ultrasonic wave, the transducers (T₁-Tₙ) of the array are activated in emission according to a respective activation delay law, so that each transducer (T₁-Tₙ) emits an elementary ultrasonic wave (El₁-Elₙ) at a respective instant as a function of said activation delay law.

The elementary ultrasonicwaves (El₁-Elₙ) combine to form the emitted wave propagating along a direction having the desired emission angle, the emission angle of the emitted wave depending on the activation delay law used.

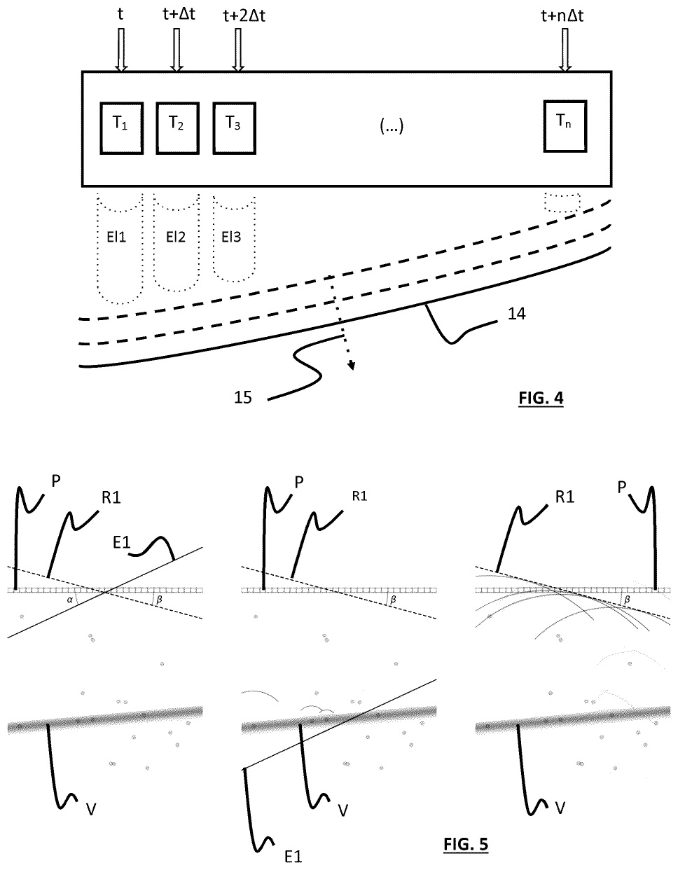

More specifically, as a function of the phase and amplitude of the excitation voltages applied to the transducers T₁-Tₙ by the control and processing unit U_{c}, it is possible to control the transducers T₁-Tₙ so that they produce elementary ultrasonic waves El₁-Elₙ combining to form an emitted ultrasonic wave 14 that propagates through the medium to be analyzed long a desired direction 15 (see FIG. 4).

This resulting emitted ultrasonic wave 14 can be emitted at different emission angles (i.e., different directions) by varying the activation instants (t, t+Δt, t+2Δt, . . . t+nΔt) of each transducer T₁-Tₙ of the array.

For example, for the generation of an emitted plane wave, all the transducers T₁-Tₙ can be activated:
simultaneously to obtain an emitted plane wave propagating at a zero emission angle relative to the array of transducers T₁-Tₙ, or
successively (as a function of an activation delay law) to obtain an emitted plane wave propagating at a non-zero emission angle relative to the array of transducers T₁-Tₙ.

### 3.3. Acquisition

After each emission of an emitted wave with a desired emission angle, the transducers (T₁-Tₙ) of the array are activated in reception for the acquisition of a time-dependent reception signal.

Each reception signal is representative of a received wave propagating along a direction with a desired reception angle.

For the acquisition of a reception signal, one solution consists in activating (in reception) simultaneously the transducers T₁-Tₙ of the array. In this case, the transducers T₁-Tₙ acquire simultaneously the reverberated signals, independently of the orientation of the received waves (i.e., independently of the directions of movement of their wave fronts).

Thus, after the emission of an emitted wave with a given emission angle, each transducer is activated simultaneously in reception to record reverberated signals representative of the reverberation by the medium of the emitted wave.

For each transducer T₁-Tₙ, a reverberated signal as a function of time t, {sᵢ(t)}_{0≤i≤n-1} is recorded.

The reverberated signals recorded by the transducers T₁-Tₙ are then summed according to a time delay law depending on the desired reception angle for the received wave.

For example, for the acquisition of a reception signal representative of a received wave having a reception angle β from the reverberated signals {sᵢ(t)}_{0≤i≤n-1} measured by the transducers T₁-Tₙ, the following summation operation is performed:

$$= {\sum }_{i = 0}^{n - 1} {a}_{i} {s}_{i} ( t - \frac{ip sin β}{c} )$$

where:
“c” represents the wave velocity in the medium,
“p” represents the pitch of the probe (i.e., deviation between the centers of two adjacent transducers in the array of transducers),
the coefficients “aᵢ” are apodization coefficients of the elements of the array of transducers.

Advantageously, it is possible to apply an apodization during the reception operation, that is to say a spatial windowing resulting in coefficients in front of the signals of each transducer.

The processing of the reverberated signal block makes it possible to “reorient” the responses recorded by the different transducers T₁-Tₙ in order to obtain the reception signals representative of the waves received at the different desired reception angles.

As indicated previously, the delay laws used for the summation of the reverberated signals recorded by the transducers of the array in order to determine the reception signals depend on the desired reception angles for the received waves associated with these reception signals.

These desired reception angles are chosen so that the bisectors of the different pairs of emitted and received waves coincide.

For example, in the case of the successive emission of first, second and third emitted waves associated respectively with first, second and third emission angles (for example equal to 0°, to −10° and to −20° respectively), then the reverberated signals recorded by the transducers are summed according to first, second and third delay laws representative of first, second and third received waves associated respectively with first, second and third reception angles (for example of 0°, of 10° and of 20°) chosen so that:
the bisector of the pair formed by the first emitted and received waves (pair α=0°, β=0°),
the bisector of the pair formed by the second emitted and received waves (pair α′=−10°, β′=10°),
the bisector of the pair formed by the third emitted and received waves (pair α″=−20°, β″=20°),
   coincide.

In other words, each pair of emitted and received waves (α, β; α′, β′; α″, β″) “shares” the same average angle

$$( \frac{α + β}{2} ; \frac{α′ + β′}{2} ; \frac{α′′ + β′′}{2} ) .$$

Thus, the delay laws used to calculate the reception signals representative of the waves received at the different reception angles are determined so that the average angles of the pairs of emitted and received waves are equal.

### 3.4. Distortion

At the end of the acquisition step, we obtain a plurality of time-dependent reception signals, each reception signal being associated with a respective pair of emitted and received waves. Typically for two emitted waves, it is possible to determine between two and several tens of received waves, making it possible to obtain between two and several tens of reception signals associated with respective pairs of emitted and received waves.

Since the average angles

$$( \frac{α + β}{2} ; \frac{α′ + β′}{2} ; \frac{α′′ + β′′}{2} )$$

of the different pairs of emitted and received waves are equal (equal to 0 in the case of a first pair α=0° and β=0°, of a second pair α′=−10° and β′=10°, and of a third pair α″=−20°, β″=20°), the reception signals associated with these different pairs are theoretically equal, to within one distortion.

Indeed, each reception signal contains information representative of the same imaged area. However, since the emission and reception angles of each pair are different, this information is not received at the same instant by the transducers, so that the different signals are time distorted.

More specifically, the emitted and received waves of each pair propagate at the same speeds through the different layers of the scattering medium. However, the distance traveled by the emitted and received waves of each pair varies as a function of their emission and reception angles. The greater the emission and reception angles of a pair, the smaller the distance traveled by the emitted and received waves of this pair to reach a depth of the medium.

Thus, the information representative of the imaged area contained in a first reception signal associated with a first pair of emitted and received waves will be time “compressed” compared to the information contained in a second reception signal associated with a second pair of emitted and received waves if the emission and reception angles of the first pair are greater than the emission and reception angles of the second pair (later reception on the transducers in the case of the second reception signal since the emitted and received waves of the first pair travel a shorter distance than the distance traveled by the emitted and received waves of the second pair).

It follows that the reception signals associated with the different pairs of emitted and received waves are of different durations, even if they contain the same information. This is illustrated in particular in FIG. 7e in which two reception signals s(t) and s′(t) each associated with a respective pair of emitted and received waves contain the same information over a different duration: the signal s(t) (associated with a pair of emitted and received waves having emission and reception angles lower than the emission and reception angles of the pair of emitted and received waves associated with the signal s′(t)) is temporally expanded relative to the signal s′(t).

To compensate for this difference in duration, the reception signals are distorted by composition with an affine function making it possible to express the reception signals over the same duration.

In particular, a homothetic change of variable is applied to each of the reception signals. This homothetic variable change consists, for each reception signal, in a mathematical time expansion or contraction.

We call “mathematical time expansion” (respectively “mathematical time contraction”) the expansion transformation (respectively contraction) when it is applied to a time signal. It makes it possible to increase or decrease the duration of the time signal without changing its overall form. In other words, we obtain a slowed down (respectively accelerated) time signal compared to the original time signal.

Thus, the analysis method comprises a distortion step (time stretching or time compression) of each of the reception signals, by composition with an affine function, to obtain distorted reception signals having an identical duration.

More specifically, for each reception signal, the distortion step can consist in applying to said reception signal an affine function whose leading coefficient is a function of the half angle difference between the emission angle and the reception angle of the emitted and received waves of the pair associated with said reception signal.

As an illustration, for each reception signal, this affine function can be a cosine function of the half angle difference between the emission angle and the reception angle of the pair of emitted and received waves associated with said reception signal. For example, in the case:
of a first reception signal associated with a pair of emitted and received waves having emission and reception angles equal to 0 (pair α=0° and β=0°), the distortion can consist in multiplying the time base by the value 1

$$( cos ( \frac{0 - 0}{2} ) = 1 ) ,$$

of a second reception signal associated with a pair of emitted and received waves having emission and reception angles equal to −10 and 10 respectively (pair α′=−10° and β′=10°), the distortion can consist in multiplying the time base by the value cos 10

$$( cos ( \frac{- 10 - 10}{2} ) = cos 10 ) ,$$

of a third reception signal associated with a pair of emitted and received waves having emission and reception angles equal to −20 and 20 respectively (pair α″=−20° and β″=20°), the distortion can consist in multiplying the time base by the value cos 20

$$( \frac{- 20 - 20}{2} ) = cos 20 ) ,$$

This makes it possible to obtain distorted (expanded or compressed) signals that theoretically all have the same duration.

As previously indicated, these transformed signals are theoretically identical since they contain information representative of the same imaged area.

In practice, since the speed of sound is not known, the actual emission and reception angles of each pair of emitted and received waves are not known. Indeed, because of the laws of refraction, the directions of the emission and reception waves are directly related to the local speed of sound of the medium. Thus, (the leading coefficient of) the affine function applied to each reception signal (which is a function of the half-difference between the emission angle and the reception angle of the pair associated with said reception signal) can be incorrect.

It is this error in the distortion applied to each reception signal that allows locally estimating the speed of sound in an area of interest.

### 3.5. Estimation

Once the signals are distorted, they are compared two by two to locally estimate the speed of sound in the region of interest. More specifically, the speed of sound of a region of interest of the medium is estimated from the evolution of a time offset between the distorted signals in said region of interest.

Particularly, the local speed of sound is obtained by a formula using a correlation of the distorted signals in pairs (i.e., the distorted signals are correlated two by two) to obtain a resulting time signal.

Since the distorted signals are complex signals containing both amplitude and phase information, the resulting time signal obtained by correlation of two considered distorted signals is also a complex signal:
the amplitude of the resulting time signal is representative of a similarity over time between the two distorted signals considered,
the phase of the resulting time signal is representative of a phase shift over time between the two distorted signals considered.

By temporally deriving the phase of the resulting time signal, a transformation coefficient between the two distorted signals considered is obtained, this transformation coefficient being representative of a difference in time scale between the two distorted signals considered.

From this transformation coefficient, it is possible to estimate the local speed of sound in the region of interest.

Thus, the step of estimating the speed of sound in the region of interest comprises the sub-steps consisting in:
determining a transformation coefficient between the distorted signals considered in pairs, said transformation coefficient being representative of a difference in time scale between the distorted signals considered in pairs,
obtaining the speed of sound in the region of interest of the medium from the transformation coefficient.

To determine the transformation coefficient, a resulting time signal is calculated by averaged correlation between the distorted signals considered in pairs, the phase of this resulting signal being representative of the phase shift (or time difference) between the distorted signals considered. The implementation of an average correlation makes it possible to temporally smooth the resulting time signal.

The transformation coefficient is then obtained by time derivation of the phase of the resulting signal.

By considering first and second reception signals associated respectively with first and second pairs of emitted and received waves according to first and second emission and reception angles, the transformation coefficient can be mathematically determined from the following formula:

$$D : = \frac{\partial ∠ 〈 \hat{s} ( t ) {\hat{s}}^{′} ( t ) * 〉}{\partial t}$$

where:
[symbol] ŝ(t) [symbol] (t)* [symbol] corresponds to the average correlation of the first and second distorted signals ŝ and ŝ′,

$$\frac{\partial ∠}{\partial t}$$

is the operator of the time derivation of the phase.

The speed of sound in the region of interest can then be obtained by solving the following formula:

$${c}_{r} = {c}_{th} \sqrt{1 - \frac{\frac{2}{{w}_{c}} D}{{{δ}_{th}^{′}}^{2} - {δ}_{th}^{2}}} ,$$

where:
“cᵣ” is the speed of sound in the region of interest,
“cₜₕ” is the theoretical speed of sound used to:
   calculate the delay laws used during the acquisition step to determine the first emission and reception angles of the first pair of emitted and received waves associated with the first reception signal
   calculate the delay laws used during the acquisition step to determine the second emission and reception angles of the second pair of emitted and received waves associated with the second reception signal,
   calculate the first affine function used during the distortion step to distort the first reception signal in order to obtain the first distorted signal,
   calculate the second affine function used during the distortion step to distort the second reception signal in order to obtain the second distorted signal,
“D” is the transformation coefficient,
“w_{c}” is the central pulse of the signals s and s′ at the depth considered,
“δ” is the half-difference between the first emission and reception angles of the first pair of emitted and received waves associated with the first reception signal,
“δ′” is the half-difference between the second emission and reception angles of the second pair of emitted and received waves associated with the second reception signal.

In summary, the step of estimating the speed of sound of a region of interest consists in estimating a local speed of sound from a transformation coefficient between time portions of interest of first and second distorted signals by derivation of the phase of the average correlation of the first and second distorted signals.

For each pair of distorted signals considered, it is thus possible to determine a resulting time signal. When several pairs are considered, several resulting time signals are obtained and can be combined to estimate a speed of sound. For example, several elementary speeds of sound can then be calculated from the several resulting time signals. The speed of sound is then estimated as the average of these several elementary speeds of sound. In another version, several transformation coefficients (unbiased by the difference of the half-differences) can be calculated and averaged, in order to obtain a robust transformation coefficient used to determine the speed of sound.

### 4. Theory Relating to the Invention

Different theoretical elements relating to the invention will now be presented in order to allow those skilled in the art to better understand the advantages associated with the method and device described above.

This technique is based on an angular approach to ultrasound imaging. For the sake of simplicity, the invention will be described with reference to the use of plane waves and of a linear probe in a two-dimensional medium.

However, any type of highly angular transmission and reception (such as plane, spiral, divergent or weakly focused waves) works. Similarly, any probe geometry can be used to generate these waves, such as the linear or curved, simple or two-dimensional probes.

The linear probe considered is composed of N ultrasonic transducers, whose acousto-electric and electro-acoustic responses are assumed to be equal and denoted h(t). A Cartesian reference frame whose origin is located at the center of the probe and whose abscissa axis is aligned with the probe is considered.

To begin, we assume that the ultrasonic probe is placed in contact with a scattering medium, characterized by its reflectivity χ(x, z), modeling the local acoustic impedance variations, in which the ultrasonic waves propagate and are reverberated. We also consider the framework of the Born approximation to the first order (i.e., we assume that the multiple scattering is negligible compared to simple scattering), and we assume that the medium has a homogeneous and known speed of sound.

### 4.1. Emission Strategy

First, at least two plane waves are emitted by the ultrasonic probe P, a first E1 in a direction α and a second in a second direction α′. The direction of a plane wave designates the angle that the plane wave front E1 makes with respect to the probe P, and therefore the abscissa axis in the considered reference frame, as illustrated in FIG. 5.

To emit a plane wave in the direction α (also called plane wave of angle α) with a linear probe S, each transducer i of the probe P is electrically excited by means of a signal e(t), by being delayed as a function of its position as follows:

$${τ}_{i} = \frac{1}{c} {x}_{i} sin α ,$$

where:
τ(x) is the delay applied to the transducer located at the position x, and
C is the speed of sound of the medium.

A plane wave is thus transmitted in the medium, such that the pressure field in the medium is of the form:

$$p ( x , z , t ) = u ( t - \frac{1}{c} ( x sin α + z cos α ) )$$

where u=e*h is the convolution between the excitation function of the transducer and its electro-acoustic response, which will be assumed to be a complex function formed by the multiplication of a Gaussian function of zero mean and standard deviation σ with a complex modulation of pulse ω_{c}:

$$u ( t ) = g ( t ) {e}^{j {ω}_{c} t} with g ( t ) \sim 𝒩 ( 0 , {σ}^{2} ) .$$

### 4.2. Reception Strategy

In a second step, the emitted plane waves are reverberated by the scattering medium, and the corresponding echoes are captured by the transducers of the probe, giving rise to signals sᵢ, i between 1 and N.

These signals are then transformed to select the signals coming from the directions β for the plane wave emitted in the direction α and β′ for the plane wave emitted in the direction α′.

To select the signals coming from the direction β, in the case of plane waves, we transform the received signals to obtain a signal representative of the reverberated plane wave of angle β. To do so, the signal sᵢ received by the transducer i is delayed as a function of its position, as in the case of the emission as follows:

$${τ}_{i} = \frac{1}{c} {x}_{i} sin β$$

These delayed signals are then multiplied by a windowing function wᵢ (potentially time-dependent) and finally summed:

$$s ( t ) = {\sum }_{i = 1}^{N} {w}_{i} {s}_{i} ( t - {τ}_{i} ) ,$$

where s is the signal obtained.

### 4.3. Interpretation of the Angular Signal and Isochronous Volume

The angular signal s obtained in the previous section, after the emission of a plane wave E1 of angle α and the reception of a plane wave R1 of angle β, has some properties for the invention.

Indeed, we can express s(t) as a function of the reflectivity of the medium as follows:

$$s ( t ) = {\int }_{x} {\int }_{z} χ ( x , z ) u ( t - \frac{1}{c} ( z cos α + x sin α + z cos ( β ) + x sin ( β ) ) ) dxdz .$$

By designating by γ=(α+β)/2 the bisector of the two directions, by δ=(α−β)/2 the half-angular deviation, and by using the change of variable ζ=z cos γ+x sin γ, we obtain after simplifications:

$$s ( t ) = {\int }_{ς} ℛχ ( γ , ζ ) u ( t - 2 \frac{ζ}{c} cos ( δ ) ) d ζ ,$$

where [symbol] (γ,ζ) is the Radon transform of the reflectivity function of the medium, assessed at the angle γ and the depth ζ defined formally as follows:

$$ℛχ ( γ , ζ ) = {\int }_{x} {\int }_{z} χ ( x , z ) dir ( x sin γ + z cos γ - ζ ) dxdz ,$$

where dir(.) designates the Dirac distribution.

This amount corresponds to the integral of the reflectivity function of the medium along the line of angle γ and depth ζ.

From the previous equation, we can deduce that if the signal is very limited in time such that we can approximate it by a Dirac distribution, we obtain directly:

$$s ( t ) = ℛχ ( γ , \frac{ct}{2 cos δ} ) .$$

Thus, the signal received at instant t is equal to the integral of the reflectivity function of the medium along the line of angle γ and depth ct/(2 cos δ) (or projection), i.e., it contains the sum of all the echoes coming from this line.

In practice, since the signal is band-limited, the reflectivity is not integrated along a simple line but along a surface V of a certain axial thickness. This surface V (or volume in a three-dimensional study) corresponds to the concept of isochronous volume introduced by Mallart and Fink.

### 4.4. Composition by an Affine Function

An important property of the angular approach results from the previous equations.

We observe that for several pairs of emission and reception angles, for example (α, β) and (α′, β′), whose bisectors γ=(α+β)/2 and γ′=(α′+β′)/2 are equal, there are instants for which the isochronous volumes are the same.

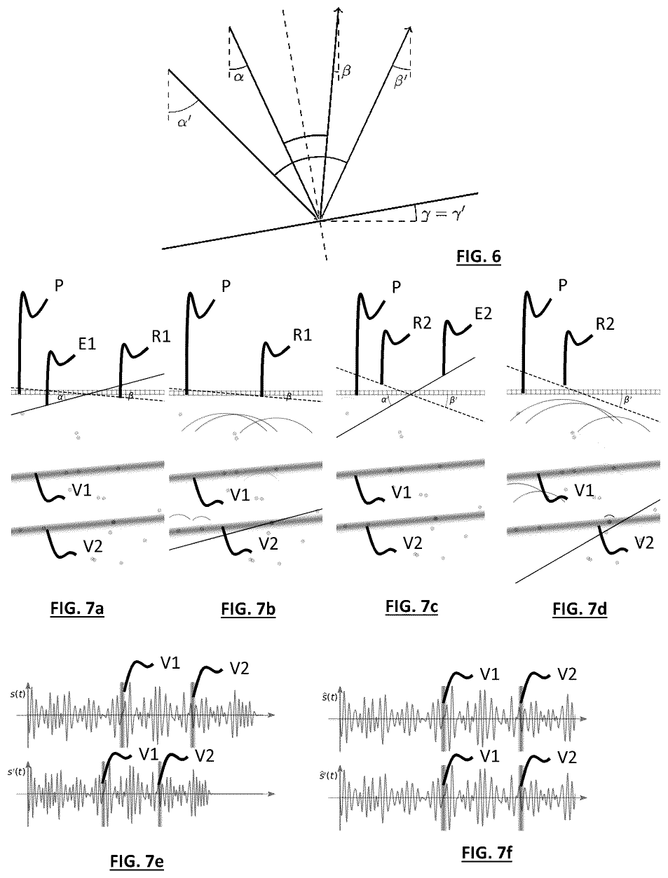

Indeed, in both cases, the latter are lines of angle γ=γ′ (of a certain thickness in the case of a band-limited pulse). Such a case is represented in FIG. 6 illustrating two pairs of emission and reception angles (α, β) and (α′, β′) sharing the same bisector γ=γ′. The two pairs give rise to the same isochronous volumes: the lines of angle γ with the horizontal.

In this case, however, the two pairs of angles give rise to different half-angular deviations δ and δ′, which gives a different link between the time t and the depth ζ:

$$ζ = \frac{ct}{2 cos δ} and {ζ}^{′} = \frac{ct}{2 cos {δ}^{′}} .$$

The difference between these time-depth relations creates an expansion phenomenon between the two signals, as represented in FIGS. 7a-7e which illustrate examples of emission and reception of two pairs E1-R1 and E2-R2 of angles (α, β) and (α′, β′) sharing the same bisector γ=γ′ in a given medium. More specifically, FIGS. 7a and 7b illustrate the emission and the propagation of a plane wave E1 of angle α and the reception R1 at an angle β, FIGS. 7c and 7d illustrate the emission and the propagation of a plane wave E2 of angle α′ and the reception R2 at an angle β′. FIG. 7e illustrates the signals s and s′ obtained in both cases, as a function of time. The instants corresponding to the coherent reception of the echoes coming from each isochronous volume V1, V2 are represented there. We observe that the signals s and s′ are identical, to within one expansion.

To compensate for this expansion effect and realign the different angular signals, the received signal s is transformed by being composed by an affine function:

$$\hat{s} ( t ) = s ( at + b ) ,$$

where:
a is the leading coefficient set to cos(δ) and
b is an offset set to 0 for the instant.

Thus, the link between time and depth becomes for ŝ: ζ=ct/2. Such a link is independent of the emitted and received angles. We note that, thanks to this composition by an affine function, all the signals obtained by having emitted and received pairs of directions having the same bisector are realigned and become equal. This effect is represented in FIG. 7f which illustrates the signals s and s′ after their expansion by a factor cos(δ).

Theoretically, by including the affine transformation in the equation defining s, we obtain:

$$s ( t ) = s ( t cos δ ) = {\int }_{ζ} ℛχ ( γ , ζ ) u ( cos ( δ ) ( t - 2 \frac{ζ}{c} ) ) d ζ .$$

We observe in this equation that the half-angular deviation δ only intervenes as a general factor in u. This effect is generally neglected because it no longer disrupts the link between time and depth but only has a slight frequency offset effect.

It is also possible to compensate for this frequency offset by filtering the received signal. In our case, this factor is neither neglected nor compensated, but taken into account in the rest of the equations.

### 4.5. Effect of an Unknown Heterogeneous Medium

Now we consider a more complex medium intended to be closer to reality. This medium is composed of several horizontal layers of varying thicknesses and speeds of sound.

Let us assume that we study the signals that correspond to a specific layer. In this layer, we have a homogeneous but unknown speed of sound. It is this speed of sound that we want to determine.

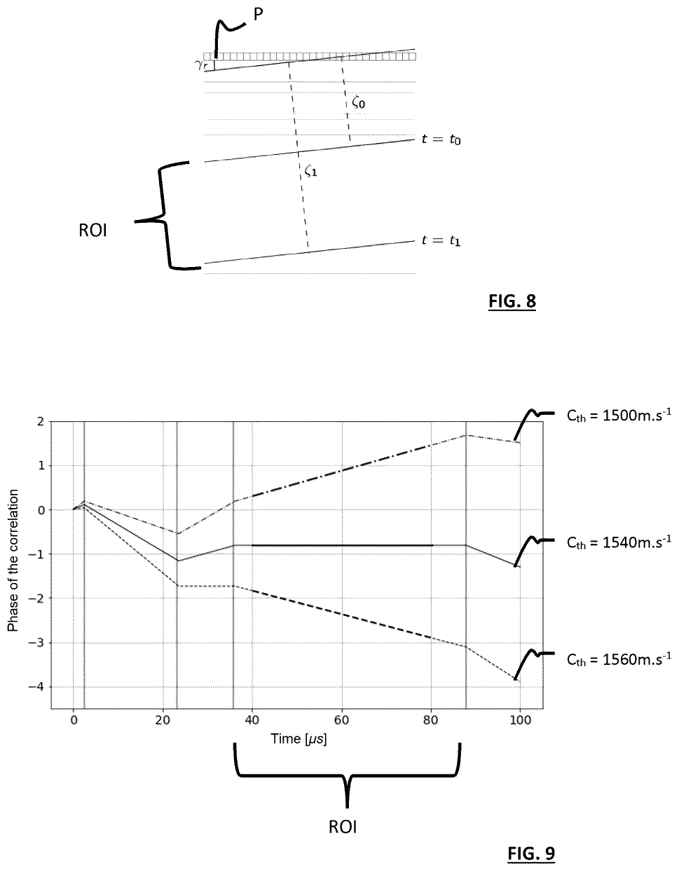

We note the time interval considered (corresponding to the layer of interest) [t₀, t₁]. We note the corresponding depths of the isochronous volumes [ζ₀, ζ₁]. Such a configuration is represented in FIG. 8.

Such a configuration is representative of different biological media such as the liver, where skin, fat and muscle layers cover the liver layer.

### 4.6. Refraction

When an emission wave passes from a first layer with a speed of sound c₁ to a second layer with a speed of sound c₂, the laws of refraction involve that the angle of the emission wave is changed.

Indeed, we have:

$$\frac{sin {α}_{1}}{{c}_{1}} = \frac{sin {α}_{2}}{{c}_{2}} ,$$

where α₁ and α₂ correspond to the angles of the emission wave in the first and second layers.

By recursion, we obtain a link between the speed of sound in any layer n and the speed of sound in this same layer with the angle and the speed of sound in the first layer:

$$\frac{sin {α}_{n}}{{c}_{n}} = \frac{sin {α}_{1}}{{c}_{1}} .$$

4.7. Emission and Reception with an Unknown Speed of Sound

As described above, to emit or receive an ultrasonic plane wave, we apply delays on the transducers.

However, the calculation of these delays assumes the knowledge of the speed of sound in the medium.

In the case of an unknown medium, we can only assume a speed of sound cₜₕ in order to emit or receive a theoretical angle αₜₕ or βₜₕ.

The form of the delay law applied to the transducers gives for the emission (the reception being similar):

$$\frac{τ ( x )}{x} = \frac{sin {α}_{th}}{{c}_{th}} .$$

Thus, by choosing τ(x), we choose not the angle but the quotient of the sine of the angle on the speed of sound.

The actual angle emitted therefore depends on the speed of sound of the first layer in the same way as for the laws of refraction.

The probe therefore behaves like an additional layer of infinitesimal thickness and speed of sound cₜₕ. We finally obtain in the layer n:

$$\frac{sin {α}_{n}}{{c}_{n}} = \frac{sin {α}_{th}}{{c}_{th}} .$$

These equations are valid for the emission and reception by reciprocity.

### 4.8. Form of the Signal Obtained

In the following, we denote:
the assumed amounts: “₋ₜₕ”, and
the actual amounts in the layer of interest: “₋ᵣ”.

Assuming a speed of sound cₜₕ, we think we are emitting and receiving plane waves of angles αₜₕ and pin, having a half-angle γₜₕ and a half-angular deviation δₜₕ.

In practice, the actual speed of sound in the layer of interest is cᵣ and the actual angular amounts are:

$$\{ \begin{matrix}{α}_{r} = arcsin ( \frac{{c}_{r}}{{c}_{th}} sin {α}_{th} ) \\ {β}_{r} = arcsin ( \frac{{c}_{r}}{{c}_{th}} sin {β}_{th} ) \\ {γ}_{r} = ( {α}_{r} + {β}_{r} ) / 2 \\ {δ}_{r} = ( {α}_{r} - {β}_{r} ) / 2\end{matrix} .$$

We deduce the signal received before composition with the affine function, between the times t₀ and t₁:

$$s ( t ) = {\int }_{ζ} ℛ χ ( {γ}_{r} , ζ ) u ( t - {t}_{0} - 2 \frac{( ζ - {ζ}_{0} )}{{c}_{r}} cos ( {δ}_{r} ) ) d ζ .$$

To realign the signals, we compose them with an affine function whose leading coefficient is the cosine of the assumed half-angular deviation δₜₕ and the bias is set to zero. We obtain:

$$\hat{s} ( t ) = {\int }_{\dot{ζ}} ℛ χ ( {γ}_{r} , ζ ) u ( t cos {δ}_{t h} - 2 \frac{( ζ - {ζ}_{c} )}{{c}_{r}} cos ( {δ}_{r} ) ) d ζ .$$

where ζ_{c}=ζ₀−(cᵣt₀)/(2 cos δᵣ) is an unknown constant corresponding to the misalignment caused by all the layers located above the layer of interest.

To ensure maximum alignment, it is possible to use the bias of the composition with the affine function to empirically compensate for this offset. This compensation is however only necessary for superficial layers with speeds of sound very different from the assumed one and is not necessary in the general case.

It will be noted that in the case of an infinitely fine signal u (Dirac), we obtain:

$$\hat{s} ( t ) = ℛ χ ( {γ}_{r} , \frac{{c}_{r} t}{2} \frac{cos {δ}_{th}}{cos {δ}_{r}} + {ζ}_{c} ) .$$

The signal is therefore independent of the angle pair used provided that δₜₕ=δᵣ and ζ_{c}=0.

In the context of this method, the constant ζ_{c} does not really interest us since it is linked to the layers that are not the one of interest.

It is the amount cᵣ/2*cos δₜₕ/cos δᵣ that interests us, since it is related only to the speed of sound in the layer of interest.

To locally estimate the speed of sound in the medium of interest, the principle is to detect this factor. Indeed, this amount informs us about the angle error made between δₜₕ and δᵣ and therefore, via the laws of refraction, about the error in the speed of sound made between cₜₕ and cᵣ.

### 4.9. Achievement of the Transformation Coefficient

To detect this transformation coefficient, a reference is necessary. To do so, we use two signals ŝ and ŝ′ generated with two pairs of angles (αₜₕ, βₜₕ) and (α′ₜₕ, β′ₜₕ) that share the same bisector γₜₕ=γ′ₜₕ.

To compare these signals, we calculate their correlation. It is the phase of this correlation that will lead us to the transformation coefficient, as represented in FIG. 9 which is a graph illustrating the phase of the correlation of two signals s and s′ corresponding to the pairs (10°, −10°) and (0°, 0°) of emitted/received waves as a function of time, in a medium composed of several layers. As represented in this figure, the speed of sound in the region of interest ROI is equal to 1,540 m/s. The different curves correspond to several speed of sound cₜₕ hypotheses, which lead to several compositions with affine functions.

In order to study this correlation, some hypotheses and simplifications are used:
it is assumed that the actual bisectors are the same (γᵣ=γ′ᵣ); this allows us to consider that the two signals correspond to the same integration volumes, to within a time offset; this approximation is close to reality since the difference between these two bisectors is generally less than 0.1°,
it is assumed that the medium is mainly composed of uniform speckle, i.e., the Radon transform of the reflectivity function can be interpreted as a probabilistic density whose achievements are independent as a function of the media or depths and whose average is uniformly equal to χₘ in the layer of interest; this allows writing that the average correlation on the media or on the depth of the Radon transforms of the reflectivity function is:

$$〈 ℛχ ( γ , ζ ) {ℛχ ( γ , {ζ}^{′} )}^{*} 〉 = {\vert {χ}_{m} \vert}^{2} dir ( ζ - {ζ}^{′} ) ,$$

where dir(.) is a Dirac and <.> designates the averaging operation over time or over different media with the same characteristics.

To simplify future calculations we define the intermediate functions:

$$\{ \begin{matrix}f ( t , ζ ) = t cos {δ}_{th} - 2 \frac{ζ - {ζ}_{0}}{{c}_{r}} cos {δ}_{r} \\ {f}^{′} ( t , ζ ) = t cos {δ}_{th}^{′} - 2 \frac{ζ - {ζ}_{0}^{′}}{{c}_{r}} cos {δ}_{r}^{′}\end{matrix} ,$$

in order to obtain:

$$\hat{s} ( t ) = {\int }_{ζ} ℛ χ ( {γ}_{r} , ζ ) u ( f ( t , ζ ) ) d ζ$$

$$and$$

$$\hat{{s}^{′}} ( t ) = {\int }_{{ζ}^{′}} ℛ χ ( {γ}_{r}^{′} , {ζ}^{′} ) u ( {f}^{′} ( t , {ζ}^{′} ) ) d {ζ}^{′} .$$

It is possible to calculate the form of the correlation between these two signals averaged over time (i.e., the depth). After a few calculations, we obtain:

$$〈 \hat{s} ( t ) {\hat{{s}^{′}} ( t )}^{*} 〉 = {\int }_{ζ} {\int }_{{ζ}^{′}} 〈 ℛχ ( {γ}_{r} , ζ ) {ℛχ ( {γ}_{r}^{′} , {ζ}^{′} )}^{*} 〉 u ( f ( t , ζ ) ) {u ( {f}^{′} ( t , {ζ}^{′} ) )}^{*} d {ζ}^{′} d ζ .$$

By using the hypotheses described above, this expression simplifies to:

$$〈 \hat{s} ( t ) {\hat{{s}^{′}} ( t )}^{*} 〉 = {\vert {χ}_{m} \vert}^{2} {\int }_{ζ} u ( f ( t , ζ ) ) {u ( {f}^{′} ( t , ζ ) )}^{*} d ζ .$$

In order to understand the influence of the expansion factor on this correlation, we develop the function u by using its expression defined at the beginning:

$$〈 \hat{s} ( t ) {\hat{{s}^{′}} ( t )}^{*} 〉 = {\vert {χ}_{m} \vert}^{2} {\int }_{ζ} g ( f ( t , ζ ) ) {g ( {f}^{′} ( t , ζ ) )}^{*} {e}^{{jw}_{e} ( f ( t , ζ ) ) - {f}^{′} ( t , ζ ) )} d ζ .$$

Here, we see that the phase of the integrated expression is representative of the difference of the intermediate functions. By definition of the intermediate functions, we see that it informs us about this expansion factor. It will therefore be the phase of this expression that will interest us.

To calculate it, we start by studying the two functions g(f(t, .)) and g(f′(t, .)). The function g being a Gaussian of law N(0, σ²) and f(t, .) and f′(t, .) being affine functions, we deduce that the two composite functions are themselves Gaussian functions according to the laws:

$$\{ \begin{matrix}g ( f ( t , \cdot ) ) ∼ 𝒩 ( \frac{{c}_{r} t}{2} \frac{cos {δ}_{th}}{cos {δ}_{r}} + {ζ}_{c} , {σ}^{2} \frac{4}{{c}_{r}^{2} {cos}^{2} {δ}_{r}} ) \\ g ( {f}^{′} ( t , \cdot ) ) ∼ 𝒩 ( \frac{{c}_{r} t}{2} \frac{cos {δ}_{th}^{′}}{cos {δ}_{r}^{′}} + {ζ}_{c}^{′} , {σ}^{2} \frac{4}{{c}_{r}^{2} {cos}^{2} {δ}_{r}^{′}} )\end{matrix} .$$

Now, the product of two Gaussians is itself a Gaussian function, whose average is:

$${μ}_{tot} = \frac{{μ}_{1} {σ}_{2}^{2} + {μ}_{2} {σ}_{1}^{2}}{{σ}_{1}^{2} + {σ}_{2}^{2}} ,$$

where μ_{1, 2} are the averages of the two multiplied Gaussian functions and σ_{1,2} their variances.

In our case, we obtain after simplifications:

$${μ}_{tot} = \frac{{c}_{r} t}{2} \frac{cos {δ}_{th} cos {δ}_{r} + cos {δ}_{th}^{′} cos {δ}_{r}^{′}}{{cos}^{2} {δ}_{r} + {cos}^{2} {δ}_{r}^{′}} + \frac{{ζ}_{c} {cos}^{2} {δ}_{r} + {ζ}_{c}^{′} {cos}^{2} {δ}_{r}^{′}}{{cos}^{2} {δ}_{r} + {cos}^{2} {δ}_{r}^{′}} .$$

By denoting G(ζ), the equivalent Gaussian of average μₜₒₜ and by noting that f(t, ζ)−f′(t, ζ) is an affine function in ζ that we will denote f(t, ζ)−f′(t, ζ)=aζ+b, we obtain:

$$〈 \hat{s} ( t ) \hat{{s}^{′}} {( t )}^{*} 〉 = {\vert {χ}_{m} \vert}^{2} {\int }_{ζ} G ( ζ ) {e}^{j {ω}_{c} ( a ζ + b )} d ζ .$$

Now, it is possible to prove by Hilbertian symmetry around μₜₒₜ that the phase of such an expression is simply:

$$∠ 〈 \hat{s} ( t ) \hat{{s}^{′}} {( t )}^{*} 〉 = {ω}_{c} ( a {μ}_{tot} + b ) .$$

By replacing μₜₒₜ, a and b, we obtain the formula after simplifications:

$$∠ 〈 \hat{s} ( t ) {\hat{{s}^{′}} ( t )}^{*} 〉 = {ω}_{c} t \frac{cos {δ}_{r} + cos {δ}_{r}^{′}}{{cos}^{2} {δ}_{r} + {cos}^{2} {δ}_{r}^{′}} ( cos {δ}_{th} cos {δ}_{r}^{′} - cos {δ}_{th}^{′} cos {δ}_{r} ) + C ,$$

where C is a constant that does not depend on time.

In this equation, C represents the offset caused by all the layers between the probe and the layer of interest, while the coefficient in front of ω_{c}t is representative of the expansion factor and depends only on the layer of interest. This is represented in FIG. 9, where we see that the amount of interest is in fact the derivative of this phase. We note for example that in the case where cₜₕ=1,540 m/s, the derivative of this phase is zero because the assumed angle deviation δₜₕ is equal to the actual angle deviation dr. For assumed speeds cₜₕ too high, the derivative will be negative because the assumed angle deviation δₜₕ is too large, and for speeds too low, the derivative will be positive.

Thus, to obtain only the information on the layer of interest, we denote D the local transformation coefficient calculated as follows:

$$D := \frac{\partial ∠ 〈 \hat{s} ( t ) {\hat{{s}^{′}} ( t )}^{*} 〉}{\partial t} = {ω}_{c} \frac{cos {δ}_{r} + cos {δ}_{r}^{′}}{{cos}^{2} {δ}_{r} + {cos}^{2} {δ}_{r}^{′}} ( cos {δ}_{th} cos {δ}_{r}^{′} - cos {δ}_{th}^{′} cos {δ}_{r} ) .$$

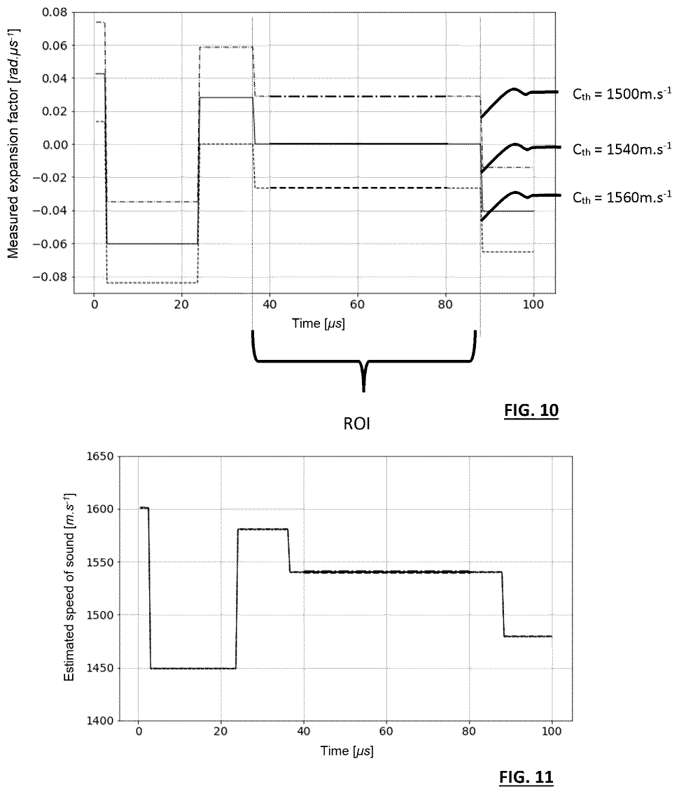

Such a measured transformation coefficient, in the case of the medium corresponding to FIG. 8, is represented in FIG. 10 which illustrates a measured expansion factor for a multilayer medium and for several speed of sound hypotheses. To use the properties of the speckle, a sliding averaging of about 20 μs was used on the latter, degrading the axial resolution.

We note once again that in the case where cₜₕ=1,540 m/s, the estimated transformation coefficient is zero. For assumed speeds cₜₕ too high, this transformation coefficient will be negative, and for speeds too low, it will be positive.

All that remains is to find an explicit formula that links the actual speed of sound to this expansion factor.

### 4.10. Achievement of the Local Speed of Sound

To obtain the local speed of sound from the local transformation coefficient D, we study the expression of D by carrying out a limited development to the second order on the angles. We obtain that δᵣ˜δₜₕ (cᵣ/cₜₕ), and in the same way δ′ᵣ˜δ′ₜₕ (cᵣ/cₜₕ), which gives:

$$D \approx \frac{{ω}_{c}}{2} ( 1 - \frac{{c}_{r}^{2}}{{c}_{th}^{2}} ) ( {{δ}_{th}^{′}}^{2} - {δ}_{th}^{2} ) .$$

We therefore obtain for the speed of sound:

$${c}_{r} = {c}_{th} \sqrt{1 - \frac{\frac{2}{{ω}_{c}} D}{{{δ}_{th}^{′}}^{2} - {δ}_{th}^{2}}} , with D := \frac{\partial ∠ 〈 \hat{s} ( t ) {\hat{{s}^{′}} ( t )}^{*} 〉}{\partial t} .$$

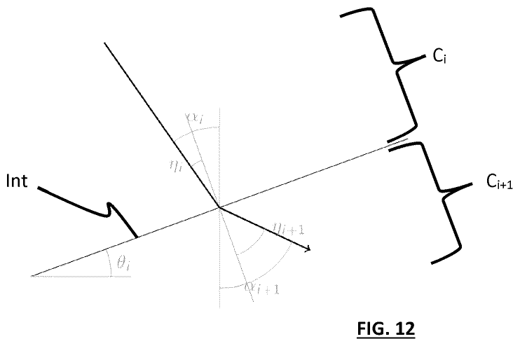

Such a measured speed of sound, in the case of the medium corresponding to FIG. 8, is represented in FIG. 11 which illustrates a speed of sound estimated from an expansion factor for a multilayer medium as a function of time.

Here we observe that the speed of sound, directly obtained from the correlation between the two signals ŝ and ŝ′, is indeed a local amount. In addition, all the assumed speed of sound cₜₕ hypotheses give very similar results (to within 10 m/s).

We note that this speed of sound is obtained here from a simulation, with only two pairs of emitted and received plane waves.

The invention presented here indeed makes it possible to locally estimate the speed of sound with only two emissions, two signals obtained (i.e., an extremely limited number of data), and an inexpensive calculation (a composition by affine function, a correlation, a transition to the phase, a derivation and the application of a direct formula).

### 4.11. Interpretation

The speed of sound obtained by the equation above is representative of a local speed of sound, not disturbed by the heterogeneities of the medium located higher than the region of interest, in the hypothesis of a medium stratified in horizontal layers.

We obtain that the calculated speed of sound will be substantially equal to the average of the speeds of sound along the isochronous volume (by limited development, error of less than 5 m/s for speeds of sound ranging from 1,400 to 1,650 m/s).

### 4.12. Central Pulse

The factor ω_{c} present in the equation above corresponds to the central pulse of the ultrasonic wave.

It can be assumed to be equal to the excitation pulse of the transducers.

However, the ultrasonic media tend to attenuate the ultrasonic signals in a non-linear manner, which causes an offset towards the low frequencies of this central pulse. To counter this effect, it is possible to measure the value of this pulse over time by using the autocorrelation of the signals.

We have indeed:

$${ω}_{c} = \frac{\partial 〈 ∠ s ( t ) {s ( t + dt )}^{*} 〉}{\partial t}$$

where the average is calculated over time or across different media.

### 4.13. Sensitivity and Choice of the Pairs of Angles

The calculation of the local speed of sound from the expansion factor depends on the choice of the angles. More specifically, it depends on the difference of the squares of the half-angular deviations. The smaller this difference, the more a slight variation in the expansion factor will involve a large variation in the estimated speed of sound. It is therefore appropriate to choose pairs of angles whose half-angular deviations are sufficiently far apart to guarantee certain stability to the technique.

Finally, we will note that the stability of the method does not depend on the depth, which is an advantage over several techniques of the state of the art, particularly for the quantification of the speed of sound in organs located several centimeters deep such as the liver.

### 4.14. Case of Non-Horizontal Layers

In practice, it is possible to want to image a medium composed of non-horizontal layers. In this case, the laws of refraction no longer apply in such a simple way.

Let us assume that a plane wave of angle αᵢ propagates from a layer i to a layer i+1, with speeds of sound cᵢ and cᵢ₊₁ and separated by an interface of angle θᵢ. Such a configuration is represented in FIG. 12 which represents the propagation of an emission wave of angle α_{I} between a first layer Cᵢ and a second layer Cᵢ₊₁ separated by an interface Int of angle θᵢ.

In this case, the laws of refraction indicate that:

$$\frac{sin {η}_{i}}{{c}_{i}} = \frac{sin {η}_{i + 1}}{{c}_{i + 1}} ,$$

where ηᵢ and ηᵢ₊₁ are the angles between the incident wave and the refracted wave and the normal to the interface. We obtain:

$$\frac{sin ( {α}_{i} - {θ}_{i} )}{{c}_{i}} = \frac{sin ( {α}_{i + 1} - {θ}_{i + 1}}{{c}_{i + 1}} .$$

The modification of this recurrence formula, valid both in emission and in reception, will disturb the local expression of α and β and therefore that of δ.

In practice, it is possible to carry out a limited Taylor development to the second order on the angles, simplifying these relations and preserving the simplified recurrence relation:

$$\frac{{δ}_{1}}{{c}_{i}} \approx \frac{{δ}_{i + 1}}{{c}_{i + 1}} .$$

In the most common case of interfaces with angles smaller than 45°, we obtain angle deviations with the simplified formula of less than 2°.

The previous theory can therefore still be applied. Of course, the larger the angles of the interfaces, the more the method described above can present a bias.

### 4.15. Value, Profile and Map

The technique presented here allows simply obtaining three types of results.

First, we can directly obtain the local speed of sound in a region of interest. To do so, a windowing function for the emission and/or reception will be chosen in order to limit the lateral extension of the waves used. On the other hand, the axial extension of the region of interest will be used to average the correlation between the two signals ŝ and ŝ′ on the one hand and to calculate a robust derivative of the phase of the correlation on the other hand.

It is also possible to obtain the profile of the local speed of sound along the bisector γ used (quasi-axial profile). To obtain such a profile, it is sufficient to measure the local speed of sound at several depths.

These first two results can be consolidated by repeating the technique for different pairs of angles sharing the same bisectors γ but different half-angular deviations δ, or even slightly different bisectors.

The results obtained with these different pairs of angles can be grouped at different steps of the calculation. For example, it is possible to average all the final speeds obtained or to directly average the correlation phases, while taking care to scale them by dividing them by the difference of the squares of the half-angular deviations.

Finally, by transmitting several pairs of angles with varied bisectors, we obtain enough spatial information to reconstruct a map, for example by measuring the local speed of sound in a multitude of regions of interest distributed in the medium. It is also possible to reconstruct a map by using an angle-based approach.

Indeed, as detailed in the interpretation paragraph, the local speed of sound obtained is actually close to the average speed of sound along the isochronous volume (typically a line of angle γ with the horizontal). Thus, Radon inversion algorithms can be used to recover a local map of the speed of sound, such as the inverse projection or other inverse problems. These algorithms can operate on the local speeds of sound obtained by the technique, or directly at earlier steps in the calculation (typically on the expansion factors scaled by being divided by the difference of the squares of the half-angular deviations).

### 5. Conclusions

The method described above differs from the prior art because it makes it possible to obtain a map and/or a local measurement of the speed of sound in a region of interest directly and with the following advantages:
it provides a local measurement in a region of interest (map or value),
it requires very few ultrasonic emissions (typically between 2 and 16) compared to other local measurement techniques (typically more than 100),
it requires a very limited amount of calculation unlike the other non-direct local measurement techniques which require inverting matrices or storing large pre-inverted matrices and applying them,
it requires a very limited amount of data derived from the ultrasonic probe (works from 2 signals where the other methods generally use 100*128 signals).

In the preceding description, the invention was described in the case of the use of plane waves. The reader will appreciate that the invention can be implemented by using other types of highly directional waves, such as spiral waves and divergent waves.

Similarly, in the preceding description, the invention was described with reference to an array of transducers T₁-Tₙ having a linear geometry. It is obvious to those skilled in the art that the array of transducers T₁-Tₙ can have other shapes such as a curved or matrix shape.

In the case of a matrix probe, therefore two-dimensional probe, the preceding method is generalized by defining two-dimensional plane, spiral or divergent waves. These plane, spiral or divergent waves are nothing other than the combination of a plane, spiral or divergent wave along one axis with a plane, spiral or divergent wave along another axis, thus giving delay laws defined in Cartesian, cylindrical or polar reference frames.

1. An analysis method for analyzing a medium from an array of transducers, said method comprising:
the acquisition of at least two reception signals each associated with a respective pair of emitted and received waves, said at least two reception signals comprising:
a first reception signal associated with a first pair of emitted and received waves along first emission and reception directions,
a second reception signal associated with a second pair of emitted and received waves along second emission and reception directions,
the first emission and reception directions being different from the second emission and reception directions, the acquisition step including, for each reception signal, the following sub-steps:
generating in a scattering medium, by transducers of the array, an emitted wave having a desired emission direction,
receiving, by transducers of the array, reverberated signals and their combinations to obtain a time reception signal representative of a received wave reflected by the scattering medium along a desired reception direction,
the distortion of said at least two reception signals by composition with a respective affine function:
the first reception signal being distorted by composition with a first affine function to obtain a first distorted signal, the first affine function depending on the first emission and reception directions,
the second reception signal being distorted by composition with a second affine function, different from the first affine function, to obtain a second distorted signal, the second affine function depending on the second emission and reception directions,
the local estimation of the speed of sound in a region of interest of the medium from the comparison of the first and second distorted signals.

2. The analysis method according to claim 1, wherein the estimation step comprises the following sub-steps:
determining a transformation coefficient between the first and second distorted signals, said transformation coefficient being representative of a difference in time scale between the first and second distorted signals,
obtaining the speed of sound in the region of interest of the medium from the transformation coefficient.

3. The analysis method according to claim 2, wherein the estimation step comprises a sub-step of calculating a resulting signal representative of an average correlation between the first and second distorted signals.

4. The analysis method according to claim 3, wherein the average correlation is obtained by at least one of the following methods:
averaging over different depths in the region of interest,
averaging over different media of the same speed of sound,
averaging over signals obtained with different emission or reception strategies,
averaging over different signals obtained by disturbing delay laws applied to the array of transducers in emission or in reception.

5. The analysis method according to claim 3, wherein the transformation coefficient is determined by derivation of a phase of the resulting signal representative of the average correlation between the first and second distorted signals.

6. The analysis method according to claim 5, wherein the transformation coefficient D is determined from the following formula:
$$D := \frac{\partial ∠ 〈 \hat{s} ( t ) {\hat{{s}^{′}} ( t )}^{*} 〉}{\partial t}$$
where: [symbol]
ŝ(t) [symbol] (t)* [symbol] corresponds to the average correlation of the first and second distorted signals ŝ and ŝ′,
$$\frac{\partial ∠}{\partial t}$$
is the operator of the time derivation of the phase.

7. The analysis method according to claim 2, wherein
the sub-step of obtaining the speed of sound in the region of interest includes the resolution of the following formula:
$${c}_{r} = {c}_{th} \sqrt{1 - \frac{\frac{2}{{ω}_{c}} D}{{{δ}_{th}^{′}}^{2} - {δ}_{th}^{2}}} ,$$
where:
“cᵣ” is the speed of sound in the region of interest,
“cₜₕ” is a theoretical speed of sound used to calculate the first and second affine functions during the distortion step,
“D” is the transformation coefficient,
“ω_{c}” is a central pulse of signals s and s′ at the considered depth,
“δ” is a half-angular difference between the first emission and reception directions of the first pair of emitted and received waves,
“δ′” is a half-angular difference between the second emission and reception directions of the second pair of emitted and received waves.

8. The analysis method according to claim 1, wherein each affine function is of the type:
$$f ( t ) = A t + B ,$$
where:
“A” is a leading coefficient depending on the half-angular difference between the emission direction and the reception direction of the pair of emitted and received waves associated with the reception signal, and
“B” is a factor of alignment of the reception signals at a desired depth corresponding to the depth of the region of interest.

9. The analysis method according to claim 8, which comprises a step of determining the alignment factor “B”, said determination step comprising the sub-steps consisting in:
acquiring two primary signals each associated with a respective pair of emitted and received waves along emission and reception directions, each primary signal representing an amplitude as a function of a time variable,
temporally widening each primary signal by application of a factor to the time variable to obtain an enlarged primary signal, the factor depending on the half-angular difference between the emission direction and the reception direction of the pair associated with the primary signal,
correlating the enlarged primary signals,
deducing from the phase of the correlation an optimal offset index B.

10. The analysis method according to claim 1, wherein for each reception signal, the sub-step of combining the reverberated signals comprises the summation of the reverberated signals according to a respective delay law, and wherein said respective delay laws are defined so that the bisectors of the directions of the emission and reception waves of each pair of emitted and received waves coincide.

11. The analysis method according to claim 7, which further comprises a step of estimating the central pulse of the signals s and s′ at the considered depth, said estimation step comprising the following sub-steps:
Estimating the central pulse of the first reception signal s from its autocorrelation,
Estimating the central pulse of the second reception signal from its autocorrelation,
Estimating a final central pulse from the average of the central pulses of each of the reception signals.
